(19) United States
(12) Patent Application Publication
(10) Pub. No.: US 2023/0210925 A1
(43) Pub. Date: Jul. 6, 2023

(54) COMPOSITION FOR CYTOTOXIC T LYMPHOCYTE (CTL) ACTIVATION

(71) Applicants: DIC Corporation, Tokyo (JP); National University Corporation Hokkaido University, Sapporo-shi (JP)

(72) Inventors: Yasuyuki Imai, Ichihara-shi (JP); Tomohiro Hirahashi, Ichihara-shi (JP); Masanori Kasahara, Sapporo-shi (JP); Aya Miyazaki, Sapporo-shi (JP); Tsukasa Seya, Sapporo-shi (JP); Misako Matsumoto, Sapporo-shi (JP); Utano Tomaru, Sapporo-shi (JP)

(73) Assignees: DIC Corporation, Tokyo (JP); National University Corporation Hokkaido University, Sapporo-shi (JP)

(21) Appl. No.: 18/008,698

(22) PCT Filed: Jul. 9, 2021
(86) PCT No.: PCT/JP2021/025995

§ 371 Date: Dec. 7, 2022

(30) Foreign Application Priority Data
Jul. 14, 2020 (JP) 2020-120315

(51) Int. Cl. A61K 35/748; A61K 39/395; A61P 17/00; A61P 37/00; A61P 35/00

(52) U.S. Cl. CPC A61K 35/748; A61K 39/3955; A61P 17/00; A61P 35/00; A61P 37/00

(57) ABSTRACT

A composition for activating cytotoxic T lymphocytes (CTLs) containing a spirulina extract is provided. A composition for CTL activation for proliferation of cytotoxic T lymphocytes (CTLs) containing a spirulina extract is provided. Moreover, a pharmaceutical composition for CTL activation or a food or a drink for CTL activation containing the composition for CTL activation is provided.

### TECHNICAL FIELD

The present invention relates to a composition for cytotoxic T lymphocyte (CTL) activation for proliferation of CTLs containing a spirulina extract and to a pharmaceutical composition for CTL activation and a food or a drink for CTL activation which contain the composition for CTL activation.

### BACKGROUND ART

Spirulina refers to cyanobacteria which are abundant in proteins, saccharides, vitamins, minerals and plant pigments.

It has been reported that the activation of natural killer (NK) cells is induced when spirulina (a spirulina extract) is orally administered (for example, NTL 1).

Here, various immunotherapies have been recently studied in a wide range. For example, studies on cancer therapies using immunotherapies have been extensively carried out.

In general, a living body originally has a mechanism of immune surveillance for removing cancer cells, and the onset of a cancer is suppressed. Various immunocompetent cells play a role in such so-called “cancer immunity”. Immunocompetent cells which attack cancer cells include cytotoxic T lymphocytes (CTLs) in addition to NK cells. Cytotoxic T lymphocytes (CTLs) recognize and attack cancer cells through various molecules which are specifically expressed in cancer cells and thus play a role in cancer immunity.

### CITATION LIST

### Non Patent Literature

NPL 1: Hirahashi T, et al. “Activation of the human innate immune system by spirulina: Augmentation of interferon gamma production and NK cytotoxicity by oral administration of spirulina.” International Immunopharmacology 2(2002)423-434.

### SUMMARY OF INVENTION

### Technical Problem

However, it has not been known so far that a spirulina extract, which is an extract of spirulina, activates cytotoxic T lymphocytes (CTLs), which specifically attack a tumor, and causes cytotoxic T lymphocyte (CTL)-dependent regression of a tumor.

An object of the invention is to provide a composition for activating cytotoxic T lymphocytes (CTLs) containing a spirulina extract.

### Solution to Problem

As a result of extensive studies on CTL-sensitive tumors and mice to which a spirulina extract was given, the present inventors have found that a spirulina extract causes spontaneous activation of CTLs, which specifically attack a tumor, and CTL-dependent regression of a tumor. The invention has been thus completed.

That is, the invention includes the following embodiments.

A composition for cytotoxic T lymphocyte (CTL) activation for proliferation of CTLs containing a spirulina extract.

The composition for CTL activation described in [1] above which is for oral use.

A pharmaceutical composition for CTL activation containing the composition for CTL activation described in [1] or [2] above.

The pharmaceutical composition for CTL activation described in [3] above which is used for treating any cancer of malignant melanoma (melanoma), non-small cell lung cancer, Hodgkin lymphoma, head and neck cancer, stomach cancer, kidney cancer and bladder cancer.

A combination of the pharmaceutical composition for CTL activation described in [3] or [4] above and an immune checkpoint inhibitor.

A method for treating any cancer of malignant melanoma (melanoma), non-small cell lung cancer, Hodgkin lymphoma, head and neck cancer, stomach cancer, kidney cancer and bladder cancer in which the pharmaceutical composition for CTL activation described in [3] or [4] above and an immune checkpoint inhibitor are used in combination.

A food or a drink for CTL activation containing the composition for CTL activation described in [2] above.

According to the invention, a composition for activating cytotoxic T lymphocytes (CTLs) containing a spirulina extract can be provided.

### BRIEF DESCRIPTION OF DRAWINGS

FIG. 1 is the outline explaining the schedule of the experiment conducted in Example 1.

FIG. 2 is a figure showing the results of the increases in MO5 tumor cells with oral administration of H₂O and a spirulina extract conducted in Example 1.

FIG. 3 is a figure showing the results of the increases in EG7 tumor cells with oral administration of H₂O and a spirulina extract conducted in Example 2.

FIG. 4 is a figure showing the results of suppression of a tumor in particular in mice in which significant tumor regression was observed of the results of mice to which a spirulina extract was orally administered in FIG. 3.

FIG. 5 is a figure showing the results of the increases in EG7 tumor cells with oral administration of H₂O, a spirulina extract or a spirulina extract + an anti-CD8β antibody conducted in Example 3.

FIG. 6 is the outline explaining the schedule of the experiment conducted in Example 4.

FIG. 7 is the outline explaining the co-culture procedures conducted in Example 4.

FIG. 8 is a figure showing the results of the percentages of dividing cells of the CFSE-labeled OT-1 cells in Example 4.

FIG. 9 is a figure showing the results of the increases in MO5 tumor cells with oral administration of H₂O and a spirulina extract conducted in Example 5.

FIG. 10 is a figure showing the results of an analysis of CD8+ T cells, CD4+ T cells and NK cells with flow cytometry conducted in Example 5.

FIG. 11 is a figure showing the results of the increases in MO5 tumor cells with oral administration of H₂O + an isotope control Ab or a spirulina extract + an anti-PD-LlAb conducted in Example 6.

### DESCRIPTION OF EMBODIMENTS

Although the composition for activating cytotoxic T lymphocytes (CTLs) of the invention is explained in detail below, the explanation of the constituent features described below shows examples as embodiments of the invention, and the invention is not specified by the contents.

### Composition for Cytotoxic T Lymphocyte (CTL) Activation

The invention relates to a composition for cytotoxic T lymphocyte (CTL) (also called killer T cell) activation for promoting activation of cytotoxic T lymphocytes (CTLs) which is used for proliferation of specific CTLs for an antigen of an animal including a human.

The composition for CTL activation of the invention contains a spirulina extract.

The composition for CTL activation of the invention can activate CTLs without addition of a specific antigen in an NK cell-insensitive, CTL-sensitive tumor and can suppress the tumor growth, as shown in the Examples below.

### Spirulina Extract

The spirulina extract is a liquid extract extracted from spirulina with a solvent such as water and ethanol or an extract obtained by concentrating or drying the liquid extract. The liquid extract used for producing the spirulina extract is not particularly limited as long as the effects of the invention are obtained, but for example, hot water can be used. In the invention, a liquid extract obtained by extraction from spirulina with hot water or an extract obtained by concentrating or drying the liquid extract can be used preferably.

The spirulina is a fine spiral alga belonging to the genus Spirulina of the family Oscillatoriales of the order Nostocales of the class Cyanophyceae and is abundant in proteins, saccharides, vitamins, minerals and plant pigments.

Examples of the spirulina used in the invention include Spirulina platensis, Spirulina maxima, Spirulina geitleri, Spirulina siamese, Spirulina major, Spirulina subsalasa, Spirulina princeps, Spirulina laxissima, Spirulina curta, Spirulina spirulinoides and the like, and those which can be artificially cultured and which are thus easily obtained and especially preferable are Spirulina platensis, Spirulina geitleri, Spirulina siamese and the like. Here, Spirulina is also called Arthrospira.

The method for obtaining the spirulina extract is not particularly restricted. Although a generally known method can be used, examples thereof include the methods for producing a liquid extract described in NTL 1 and JPH8-9940A. More specifically, the production method described below can be used.

### Production Method of Spirulina Extract

The spirulina extract can be produced by extracting from a spirulina alga with hot water at a temperature exceeding 100° C., adjusting the pH of the liquid extract to a specific acidic condition, then removing the insoluble fraction and thus obtaining a spirulina liquid extract.

When the spirulina extract is used in a liquid state, the spirulina liquid extract obtained in the above manner can be used directly. The spirulina liquid extract, however, can also be used as powder after concentration or drying. The spirulina extract in the invention may be an extract produced by concentrating or drying a spirulina liquid extract.

As the spirulina, commercially available spirulina may be used, or cultured spirulina may also be used. Moreover, raw spirulina may be used, or one obtained by drying raw spirulina may also be used.

The culture method for culturing spirulina may be conducted according to a general method which is used for culturing cyanobacteria. For example, spirulina can be cultured and proliferated in the open air under basic conditions.

The spirulina obtained by culturing (also called a spirulina alga below) can be used directly, or cultured spirulina may be collected with filter cloth or filter paper, washed with water and then suspended in water to obtain a suspension. Moreover, the spirulina may be a wet alga obtained by concentrating a culture solution or a suspension, a dried alga obtained by drying the wet alga by freeze-drying, spray drying or the like or powder of the dried alga.

The spirulina alga used for the hot-water extraction may be any of a wet alga, a freeze-dried alga, a spray-dried alga, a crushed alga and the like. To obtain a crushed alga, for example, crushing treatment of an alga by a general method, for example, a high-pressure pressing method or the like using a French press in an industrial case, may be used.

Next, the hot-water extraction operation is explained. For example, a spirulina alga processed as described above is suspended in advance in an extraction solvent such as distilled water in a pressurized container. The suspension concentration is not particularly restricted but is desirably 1 to 20 mass% based on the solvent considering the extraction efficiency, the costs for recovery and the like. The extraction solvent may be tap water but is preferably distilled water considering that the liquid extract is applied as a food material. The extraction temperature is a temperature exceeding 100° C., preferably 105° C. to 140° C., more preferably 110 to 130° C. The pressure during the extraction is preferably 1.0 to 2.5 atmospheric pressure. A stirring operation may be conducted but does not have to be conducted during the extraction, but a stirring operation is desirably conducted for the thermal efficiency. The extract amount increases as the extraction period becomes longer, but the extraction period may be generally 0.5 to 4 hours considering the efficiency.

Next, the algal residue and protein aggregated through thermal denaturation are removed from the algal residue suspension (pH = around 6.8 to 7.0) after such extraction operation. For example, an operation such as centrifugation and filtration of the suspension may be conducted, and a supernatant is obtained by the operation. The supernatant, however, still contains a large amount of dissolved protein, and the dissolved protein is further removed more preferably. To further remove the dissolved protein, the pH of the liquid extract is preferably adjusted to an acidic condition of the isoelectric point of the protein or lower by adding an acid to the suspension. As a result, the protein can be aggregated, and the aggregated protein can be separated by centrifugation, filtration or the like, thereby obtaining a spirulina extract. Alternatively, without removing the algal residue and the like in the beginning, the algal residue and the aggregated protein may be separated similarly by centrifugation, filtration or the like after adjusting the pH of the algal residue suspension after the extraction operation to the isoelectric point of the protein or lower in the above manner. By removing the aggregated protein, a spirulina extract containing polysaccharides at a high yield can be obtained.

The acidic condition of the isoelectric point of the protein or lower is preferably pH 4.5 or less, more preferably pH 3.75 to 4.25 because the aggregation and the precipitate formation of the protein are the greatest, further preferably pH 4.0.

The acid added for the pH adjustment may be sulfuric acid or hydrochloric acid, but considering the practical operation step and the use as a food material, an organic acid such as citric acid and malic acid is preferably used rather than an inorganic acid.

### Promotion of Cytotoxic T Lymphocyte (CTL) Activation

As shown in the Examples below, a spirulina extract proliferated CTLs without the infusion of an exogenous tumor-associated antigen (TAA) in an NK cell-insensitive, CTL-sensitive tumor. Moreover, the spirulina extract suppressed the growth of an NK cell-insensitive, CTL-sensitive tumor as shown in the Examples below. When a tumor releases essentially sufficient TAA, the spirulina extract can affect the activation of CTLs and induce tumor regression.

The composition for CTL activation of the invention can be used effectively for a pharmaceutical composition or a food or a drink for the purpose of CTL activation.

### Specification of Composition for CTL Activation

The composition for CTL activation of the invention can be applied to a pharmaceutical composition for CTL activation used for proliferating CTLs.

Moreover, the composition for CTL activation of the invention can be applied to various foods and drinks with labeling as a food or a drink for CTL activation according to the need for proliferating CTLs.

For example, the composition can be used for health foods, foods with function claims, food for special dietary uses, nutritional supplements, supplements, foods for specified health uses or the like. A part of the foods are foods for which labeling with a function is permitted and thus can be distinguished from general foods.

The form of the composition for CTL activation of the invention and a pharmaceutical composition for CTL activation or a food or a drink for CTL activation containing the composition for CTL activation (the composition for CTL activation, the pharmaceutical composition and the food or the drink are also together called “the compositions and the food or the drink for CTL activation according to the invention” below) is not particularly restricted, can be appropriately selected according to the purpose and can be, for example, solid, liquid, gel or the like. Moreover, for example, the form can be tablets (including chewable tablets and the like), capsules, a drink, jelly, granules or the like.

To the pharmaceutical composition for CTL activation containing the composition for CTL activation of the invention, an ingredient for formulation or the like such as a carrier, a binder, a stabilizing agent, an excipient, a diluent, a pH buffer, a disintegrant, a solubilizer, a solubilizing agent and an isotonic agent which are pharmaceutically acceptable and generally used can be added. The pharmaceutical composition for CTL activation of the invention can be for oral use or parenteral use but is more preferably for oral use. For oral use, a generally used form of administration, for example, a dosage form of tablets, powder, granules, capsules, a syrup, a suspension or the like, can be used. For parenteral use, a generally used form of administration, for example, injection (subcutaneous injection, intravenous injection, intramuscular injection or the like) in the dosage form of a solution, an emulsion, a suspension or the like, nasal administration in the form of a spray preparation or the like, can be used.

The compositions and the food or the drink for CTL activation according to the invention can be prepared according to a general method by appropriately combining the spirulina extract with an additive such as an excipient, a binder, a disintegrant, a lubricant, a preservative, an antioxidant, an isotonic agent, a buffer, a coating agent, a corrigent, a solubilizing agent, a base, a dispersing agent, a stabilizing agent and a colorant, when the form thereof is, for example, tablets.

The excipient may be starch or a derivative thereof (dextrin, carboxymethyl starch or the like), cellulose or a derivative thereof (methylcellulose, hydroxypropyl methylcellulose or the like), a saccharide (lactose, sucrose, glucose, trehalose or the like), citric acid or a salt thereof, malic acid or a salt thereof, ethylenediaminetetraacetic acid or a salt thereof.

The binder may be starch or a derivative thereof (gelatinized starch, dextrin or the like), cellulose or a derivative thereof (ethyl cellulose, sodium carboxymethylcellulose, hydroxypropyl methylcellulose or the like), gum arabic, traganth, gelatin, a saccharide (glucose, sucrose or the like), ethanol or the like.

The disintegrant may be starch or a derivative thereof (carboxymethyl starch, hydroxypropyl starch or the like), cellulose or a derivative thereof (sodium carboxymethylcellulose, crystalline cellulose, hydroxypropyl methylcellulose or the like), a carbonate (calcium carbonate, calcium hydrogen carbonate or the like), traganth, gelatin, agar or the like.

The lubricant may be stearic acid, calcium stearate, magnesium stearate, talc, titanium oxide, calcium hydrogen phosphate, dried aluminum hydroxide gel, a sucrose fatty acid ester, edible oil or fat or the like.

The preservative may be a paraoxybenzoate ester, a sulfite (sodium sulfite, sodium pyrosulfite or the like), a phosphate (sodium phosphate, calcium polyphosphate, sodium polyphosphate, sodium metaphosphate or the like), dehydroacetic acid, sodium dehydroacetate, glycerol sorbate, a saccharide or the like.

The antioxidant may be a sulfite (sodium sulfite, sodium hydrogen sulfite or the like), erythorbic acid, L-ascorbic acid, cysteine, thioglycerol, butylated hydroxyanisole, dibutyl hydroxy toluene, propyl gallate, ascorbyl palmitate, dl-α-tocopherol or the like.

The isotonic agent may be sodium chloride, sodium nitrate, potassium nitrate, dextrin, glycerin, glucose or the like.

The buffer may be sodium carbonate, hydrochloric acid, boric acid, a phosphate (sodium hydrogen phosphate or the like) or the like.

The coating agent may be a cellulose derivative (hydroxypropyl cellulose, cellulose acetate phthalate, hydroxypropyl methylcellulose phthalate or the like), shellac, polyvinylpyrrolidone, a polyvinyl pyridine (poly-2-vinylpyridine, poly-2-vinyl-5-ethylpyridine or the like), polyvinyl acetyl diethyl amino acetate, polyvinyl alcohol phthalate, methacrylate/methacrylic acid copolymer or the like.

The corrigent may be a saccharide (glucose, sucrose, lactose or the like), sodium saccharin, a sugar alcohol or the like.

The solubilizing agent may be ethylene diamine, nicotinamide, sodium saccharin, citric acid, a citrate, sodium benzoate, polyvinylpyrrolidone, a polysorbate, a sorbitan fatty acid ester, glycerin, polypropylene glycol, benzyl alcohol or the like.

The base may be fat (lard or the like), plant oil (olive oil, sesame oil or the like), animal oil, lanolin acid, Vaseline, paraffin, resin, bentonite, glycerin, glycol oil or the like.

The dispersing agent may be gum arabic, traganth, a cellulose derivative (methylcellulose or the like), sodium alginate, a polysorbate, a sorbitan fatty acid ester or the like.

The stabilizing agent may be a sulfite (sodium hydrogen sulfite or the like), nitrogen, carbon dioxide or the like.

The spirulina extract contents of the compositions and the food or the drink for CTL activation according to the invention differ with the conditions such as the kind, the components and the form of the pharmaceutical product or the food and may be appropriately selected, but, for example, the spirulina extract content of the pharmaceutical product in liquid form or the drink in the total mass in terms of the dry weight of the spirulina extract is preferably 0.1 mass% or more, more preferably 1 mass% or more. Moreover, the spirulina extract content of the pharmaceutical product in liquid form or the drink in the total mass in terms of the dry weight of the spirulina extract is preferably 20 mass% or less, more preferably 10 mass% or less.

Moreover, for example, the spirulina extract content of the pharmaceutical product or the food in the total mass in terms of the dry weight of the spirulina extract is preferably 0.1 mass% or more, more preferably 1 mass% or more. Further, the spirulina extract content of the pharmaceutical product or the food in the total mass in terms of the dry weight of the spirulina extract is preferably 30 mass% or less, more preferably 20 mass% or less.

Furthermore, in particular, when the form of the compositions and the food or the drink for CTL activation according to the invention is tablets, for example, the spirulina extract content of the tablet in the total mass in terms of the dry weight of the spirulina extract is preferably 20 mass% or more, more preferably 50 mass% or more. Moreover, the spirulina extract content of the tablet in the total mass in terms of the dry weight of the spirulina extract is preferably 95 mass% or less, more preferably 80 mass% or less.

In the invention, the intake of the spirulina extract is not particularly limited and is appropriately selected depending on the kind, the components or the like of the pharmaceutical product or the food or the drink, but for example, the intake per one adult per day in terms of the dry weight of the spirulina extract is preferably 0.5 g or more, more preferably 1 g or more and is preferably 6 g or less, more preferably 4 g or less.

### Pharmaceutical Composition for CTL Activation

The composition for CTL activation of the invention acts effectively on many diseases which require CTL activity. For example, the composition can be used as an agent for treating a tumor. In particular, when the tumor is an NK cell-insensitive, CTL-sensitive tumor, the composition can suppress the growth of the tumor and can be used effectively as an agent for preventing or treating the cancer.

Accordingly, the composition for CTL activation of the invention is an effective anticancer therapeutic agent for a tumor which can be regressed in a CTL-dependent manner, of the tumors described below.

The type of cancer to be treated may be, for example, malignant melanoma (melanoma), non-small cell lung cancer, Hodgkin lymphoma, head and neck cancer, stomach cancer, kidney cancer, bladder cancer or the like.

### Combination With Immune Checkpoint Inhibitor

The composition for CTL activation of the invention or the pharmaceutical composition for CTL activation containing the composition for CTL activation may further be combined with an immune checkpoint inhibitor to produce a combination.

The immune checkpoint inhibitor is not particularly restricted as long as the effects of the invention are exhibited, but an example thereof is a PD-1 pathway inhibitor.

The PD-1 pathway inhibitor refers to a drug which inhibits PD-1/PD-1 ligand pathway. The PD-1 pathway inhibitor is not particularly limited as long as the PD-1 pathway inhibitor is a drug which inhibits PD-1/PD-1 ligand pathway, but examples thereof include an anti-PD-1 antibody, an anti-PD-L1 antibody, an anti-PD-L2 antibody and the like.

When the PD-1 pathway inhibitor is used, the process in which PD-1 binds to PD-L1 (PD-1 ligand) or PD-L2 (PD-2 ligand) is inhibited, and the state in which the functions of immune cells are suppressed is solved. An anticancer action is exhibited by activating the autoimmunity, and the tumor growth is suppressed.

Examples of the anti-PD-1 antibody include nivolumab (Opdivo (registered trademark)), REGN-2810, pembrolizumab (Keytruda (registered trademark)), PDR-001, BGB-A317, AMP-514 (MEDI0680), BCD-100, IBI-308, JS-001, PF-06801591, TSR-042 and the like.

Examples of the anti-PD-L1 antibody include Atezolizumab (RG7446, MPDL3280A), Avelumab (PF-06834635, MSB0010718C), Durvalumab (MEDI4736), BMS-936559, CA-170, LY-3300054, FAZ053 and the like.

The spirulina extract can be used in combination with an anti-PD-L1 antibody as shown in the Examples below and suppressed the growth of an NK cell-insensitive, CTL-sensitive tumor also in an experiment using in combination with an anti-PD-L1 antibody. Thus, the PD-1 pathway inhibitor such as the anti-PD-L1 antibody can be used in combination with the spirulina extract for the purpose of suppressing the growth of a CTL-sensitive tumor.

### Food or Drink for CTL Activation

The composition for CTL activation of the invention can be used as a component of a product such as a drink, a food and a food or drink additive.

Examples of the drink include water, soft drinks, fruit juices, milk beverages, alcoholic drinks, non-alcoholic drinks, sport drinks, nutritional drinks and the like.

The food may be bread, noodle, rice, tofu, a dairy product, soy sauce, miso, confectionery or the like.

Such a food or a drink can be prepared by a general method.

The food or the drink of any of various forms can be prepared according to a general method by appropriately combining the spirulina extract according to the invention with any food material or with another active ingredient, an additive acceptable for foods (for example, a solvent, a softener, oil, an emulsifier, an antiseptic, an acidulant, a sweetener, a bittering agent, a flavoring agent, a pH-adjusting agent, a surfactant, a stabilizer, a colorant, a UV absorber, an antioxidant, a moisturizer, a thickener, a sticking agent, a dispersing agent, a fluidity improver, a foaming agent, a wetting agent, an aroma, a seasoning, a flavor-adjusting agent or the like) or the like.

The food or the drink for CTL activation of the invention may be labeled with “enhancing CTLs”, “preventing oncogenesis through CLT activation” or the like as a food or a drink for the purpose of proliferating CTLs.

### EXAMPLES

Although the invention is further explained in detail referring to Examples below, the scope of the invention should not be limited to the Examples.

### Materials

### Spirulina Extract

Spirulina platensis was proliferated in an outdoor culture pond under basic conditions (pH 11). Next, 50 g of algal powder obtained by spray-drying the proliferated Spirulina platensis was suspended in 500 mL of distilled water in an autoclave, and extraction was conducted for an hour at an extraction temperature of 120° C. by adjusting the pressure.

The pH of the liquid extract was adjusted to 4.0 with citric acid. The algal residue and the protein (insoluble fraction) were removed therefrom through centrifugation, and a spirulina extract which was a spirulina hot-water extraction liquid was thus obtained.

To the mice, 200 µL of the spirulina extract (corresponding to 96 mg of the spirulina powder) was administered at a time.

### Low Endotoxin Ovalbumin (OVA)

OVA manufactured by FUJIFILM Wako Pure Chemical Corporation was used.

### Dulbecco’s Modified PBS (D-PBS)

Dulbecco’s modified PBS (D-PBS) used for the substrate solution or for injection to the mice was of the grade which did not contain endotoxin (manufactured by Merck Millipore).

### Anti-PD-L1 Antibody (Ab) (Clone; 10F.9G2)

The antibody was purchased from Bio X Cell.

### Rat IgG2b Isotype Control Antibody (Ab) (LTF-2)

The antibody was purchased from Bio X Cell. LTF-2 is useful as an isotype control for a rat IgG2b antibody.

### Pam2CSK4

2,3-Bis(palmitoyloxy)propyl-Cys-Ser-Lys-Lys-Lys-Lys (Pam2CSK4) was synthesized by Biologica Co. Ltd.

### 7Aad

7-Aminoactinomycin D (7AAD, a cell survival solution) for staining dead cells was purchased from BD Biosciences.

### Mice

Inbred wild-type C57BL/6 mice were purchased from CLEA Japan, Inc. The mice were maintained under conditions free of specific pathogens, and 7- to 11-week-old mice were used.

OT-1 mice are mice with forced expression of a T cell receptor for specifically recognizing the OVA antigen.

### OVA-Expressing B16 Melanoma Cells (MO5)

The OVA-expressing B16 melanoma cells (MO5) were cultured in RPMI 1640 (manufactured by Thermo Fisher Scientific) supplemented with 10% heat-inactivated fetal bovine serum (FBS, purchased from GE Healthcare Life Sciences), 100 IU penicillin/100 µg/mL streptomycin (manufactured by Thermo Fisher Scientific) and 100 µg/mL G418 (manufactured by Roche).

### OVA-Expressing EL4 Lymphoma Cells (EG7)

The OVA-expressing EL4 lymphoma cells (EG7) (ATCC (registered trademark) CRL-2113TM) were purchased from ATCC.

The OVA-expressing EL4 lymphoma cells (EG7) were cultured in RPMI 1640 supplemented with 10% heat-inactivated FBS, 10 mM HEPES (manufactured by Thermo Fisher Scientific), 1 mM sodium pyruvate (manufactured by Thermo Fisher Scientific), 55 µM 2-mercaptoethanol (manufactured by Thermo Fisher Scientific), 100 IU penicillin/100 µg/mL streptomycin and 500 µg/mL G418.

### Example 1

To C57BL/6 mice, MO5 tumor cells (1×10⁶ cells/200 µL D-PBS per animal) were subcutaneously injected to the shaved backs of the mice.

The tumor sizes were measured using calipers and determined by the following equation.

$${\text{Tumor volume (mm}}^{3} ) = 0.52 \times ( \text{Long Diameter)} \times {\text{(Short Diameter)}}^{2}$$

When the tumor volume reached about 200 mm³, oral administration of 200 µL of the spirulina extract or H₂O with a feeding tube was started.

The oral administration of H₂O or the spirulina extract to the mice was carried out every two or three days from the day after the tumor injection (transplantation).

To the group which received the first OVA stimulation, 100 µg of OVA protein was subcutaneously injected around the tumor on day 1 and day 8 after the tumor injection.

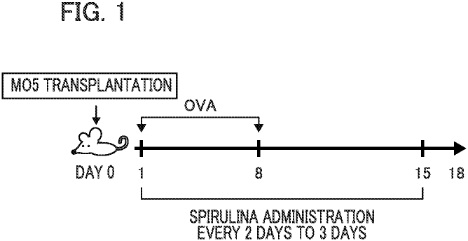

The schedule of the experiment is shown in FIG. 1.

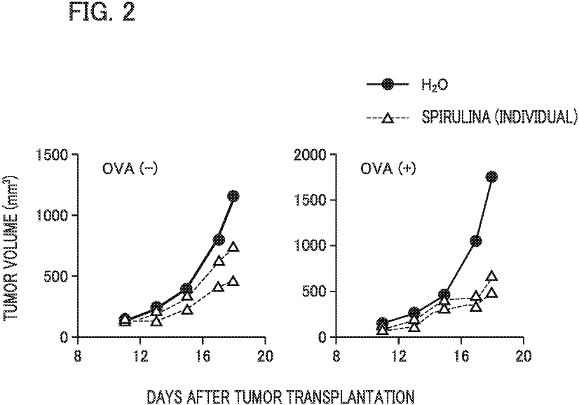

The results are shown in FIG. 2.

FIG. 2 is a figure showing the results of the increases in the MO5 tumor with the oral administration of H₂O or the spirulina extract. The data shown in the figure show the averages of the administration group for the administration of H₂O and show the results of each mouse for the administration of the spirulina extract.

The MO5 tumor is a tumor which is sensitive to both NK cells and CTLs.

As shown in FIG. 2, regardless of with or without the subcutaneous injection of OVA, mice in which the tumor regression was promoted were observed of the mice to which the spirulina extract was administered.

### Example 2

To C57BL/6 mice, EG7 lymphoma cells (2×10⁶ cells/200 µL D-PBS per animal) were subcutaneously injected to the shaved backs of the mice.

The tumor sizes were measured using calipers and determined by the following equation.

$${\text{Tumor volume (mm}}^{3} ) = 0.52 \times ( \text{Long Diameter)} \times {\text{(Short Diameter)}}^{2}$$

When the tumor volume reached about 200 mm³, oral administration of 200 µL of the spirulina extract or H₂O with a feeding tube was started.

The oral administration of H₂O or the spirulina extract to the mice was carried out every two or three days from the day after the tumor injection.

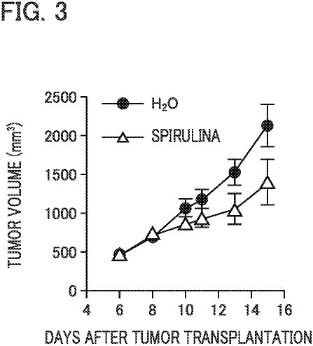

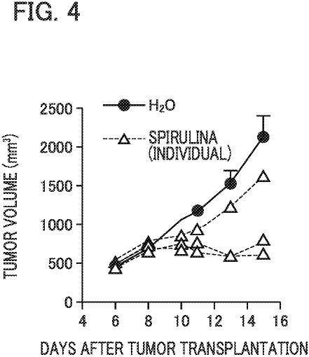

The results are shown in FIG. 3 and FIG. 4.

FIG. 3 shows the tumor growth in the mice to which H₂O or the spirulina extract was orally administered, and the data are the averages of the groups (n=5). FIG. 4 shows the results of the tumor suppression in mice in which the tumor regression was especially remarkably observed of the mice to which the spirulina extract was orally administered.

The EG7 tumor is a tumor which is insensitive to NK cells and sensitive to CTLs.

As shown in FIGS. 3 and 4, mice in which the tumor regression was promoted were observed of the mice having the EG7 tumor to which the spirulina extract was administered.

### Example 3

An experiment which was a mouse experiment conducted under similar conditions to those of Example 2 and in which an anti-CD8β antibody was further intraperitoneally injected to mice to which the spirulina extract was orally administered was conducted.

To cause CD8β cell depletion, 15 µL of hybridoma ascites containing an anti-CD8β monoclonal antibody (clone: H35.17-2) was intraperitoneally (i.p.) injected to the mice one day before the administration of the spirulina extract. Ascites generally contains 10 to 15 mg/mL IgG.

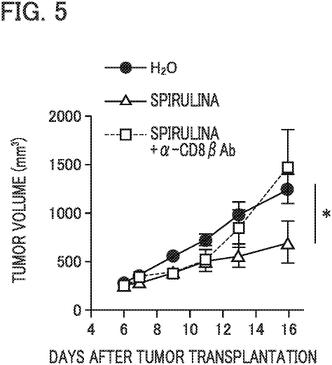

The results are shown in FIG. 5.

The results show that, while the tumor growth was suppressed in response to the administration of the spirulina extract in the mice of the spirulina extract administration group, the tumor growth was recovered through the depletion of CD8β cells in the mouse group to which the spirulina extract and the anti-CD8β antibody were administered. As a result, it was found that CTLs involved in the suppression of the tumor growth in the experimental system.

This means that it was found that, in the mice having the EG7 tumor to which the spirulina extract was administered, delay in the tumor growth with proliferation of CD8+ CTLs was induced.

### Example 4

To C57BL/6 mice, 200 µL of H₂O or the spirulina extract per animal was orally administered three times in seven days (days 0, 2 and 4).

To the group which received the first OVA stimulation, 100 µg of OVA protein was subcutaneously injected to the shaved backs of the mice on day 1.

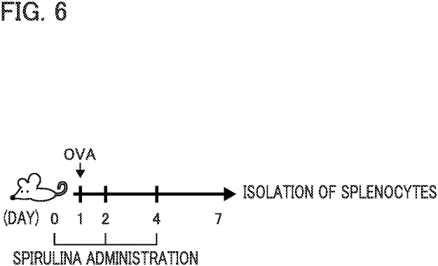

The schedule of the experiment is shown in FIG. 6.

Next, by treating with collagenase D (manufactured by Roche Diagnostics), splenocytes were isolated from the spleens of the mice to which H₂O or the spirulina extract was orally administered.

CD11c+ DC cells were isolated from the splenocytes with CD11c-microbeads (manufactured by Miltenyi Biotec) while blocking with an anti-CD16/CD32 antibody (Ab).

OT-1 T cells were isolated from the spleen of an OT-1 mouse with CD8 microbeads (manufactured by Miltenyi Biotec).

The isolated OT-1 CD8+ T cells were labeled with 1 µM of carboxyfluorescein diacetate succinimidyl ester (CFSE, manufactured by Thermo Fisher Scientific) for 10 minutes at 37° C.

To wells of a U-bottom 96-well plate, 1×10⁵ cells of the CD11c+ DC cells were seeded three times each and incubated at 37° C. regardless of with or without 2.5 µg/mL OVA.

After four hours, an equivalent amount of the CFSE-labeled OT-1 CD8+ T cells containing 5×10⁴ cells were added to the wells, and the cells were co-cultured at 37° C. for 62 hours.

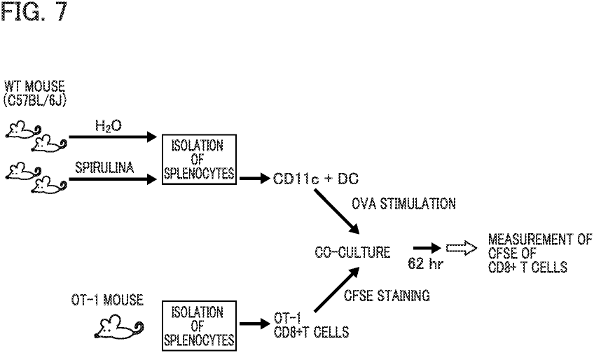

The co-culture procedures are shown in FIG. 7.

The cells were stained with an anti-CD8α antibody, an anti-CD3 antibody and an anti-TCR Vβ5.1, 5.2 antibody. The dead cells were excluded by 7AAD staining.

The proliferation of OT-1 was evaluated by the attenuation of CFSE by FACS AriaII (manufactured by BD Biosciences) and Flowjo software (manufactured by Tree Star).

In the experimental system, dendritic cells (DCs) obtained from a mouse to which the spirulina extract has been administered and T cells obtained from another mouse are co-cultured, and thus it can be observed whether the T cells are activated by the dendritic cells obtained from the mouse to which the spirulina extract has been administered.

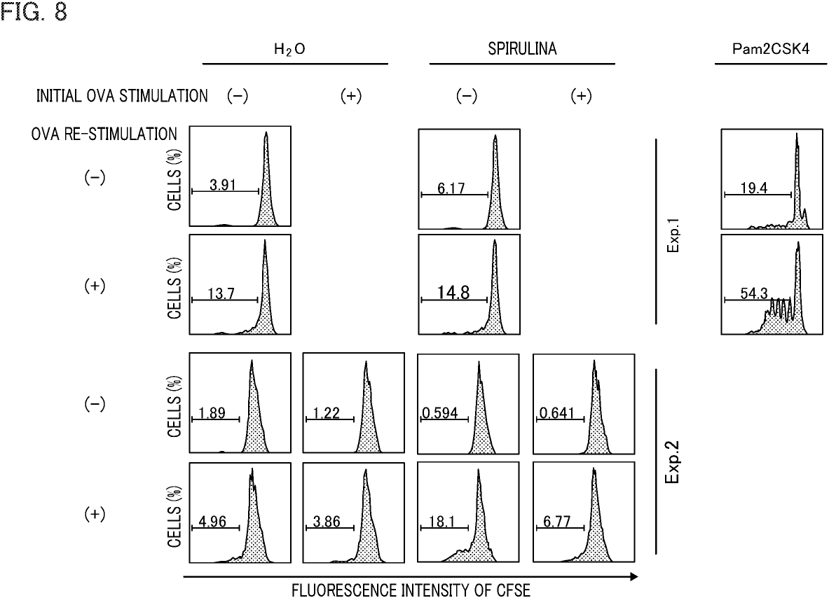

The results are shown in FIG. 8.

The graphs in FIG. 8 show the percentages of dividing cells of the CFSE-labeled OT-1 cells.

The data shown in FIG. 8 show the results of the co-culture of the T cells restimulated with OVA with or without OVA and the CD11c+ DCs.

In FIG. 8, Exp. 1 and Exp. 2 show the typical results of two independent experimental systems.

In FIG. 8, the results of a sample co-cultured with Pam2CSK4-stimulated CD11c+ DCs are also shown as a comparative experiment of a positive control.

From the results of FIG. 8, it can be seen that OVA-specific CTLs were induced, independently from the exogenous OVA administration, by the spleen DCs of the mice to which the spirulina extract was administered.

The experimental results were observed in an in vitro assay using a mixture of spleen CD11c+ DCs and OT-1 CD8+ T cells.

As shown in Exp. 2, the DCs of a spirulina-treated mouse induced the proliferation of CD8+ T cells after the re-stimulation with OVA. This occurred regardless of the first OVA stimulation.

### Example 5

In an experimental system similar to that of Example 1 in which MO5 tumor cells (1×10⁶ cells/200 µL D-PBS per animal) were subcutaneously injected to the shaved backs of C57BL/6 mice, oral administration of H₂O or the spirulina extract was conducted on day 6 and day 8 after the tumor transplantation.

The mice were slaughtered on day 11 after the tumor transplantation.

For analyzing intratumor immune cells, the tumor tissues were finely minced and treated for 15 minutes with 0.05 mg/mL collagenase I (manufactured by Sigma-Aldrich), 0.05 mg/mL collagenase IV (manufactured by Sigma-Aldrich), 0.025 mg/mL hyaluronidase (manufactured by Sigma-Aldrich) and 0.01 mg/mL DNase I (manufactured by Roche Diagnostics) in Hanks’ Balanced Salt Solution (manufactured by Sigma-Aldrich) at 25° C.

After the enzymatic treatment, the erythrocytes were lysed with ACK buffer.

Tumor infiltrating CD8+ T cells, CD4+ T cells and NK cells were stained with antibodies and analyzed with FACS AriaII (manufactured by BD Biosciences) and Flowjo software (manufactured by Tree Star).

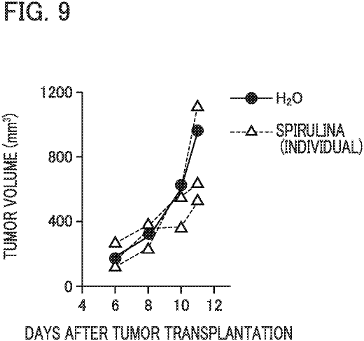

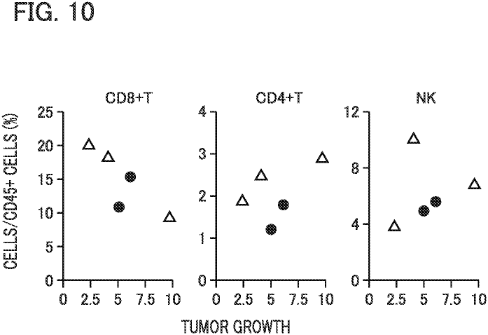

The results are shown in FIG. 9 and FIG. 10.

FIG. 9 shows the results of the increases in the tumor cells with the oral administration of H₂O and the spirulina extract.

In FIG. 9, the data of the H₂O administration group are the averages (n=2), and the data of the spirulina extract administration group are shown with individual spots (n=3).

FIG. 10 shows the results of the analysis of CD8+ T cells, CD4+ T cells and NK cells with flow cytometry in the above manner. FIG. 10 shows the relations between the percentage of the immune cells of CD8+ T cells, CD4+ T cells and NK cells in the CD45+ cells in the tumor and the ratio of the tumor growth.

As shown in FIG. 9 and FIG. 10, the CD8+ T cells in the tumor increased in the MOS-having mice to which the spirulina extract, which causes suppression of the tumor growth, was orally administered, and the percentage of the CD8+ T cells in the tumor clearly increased with the regression of the tumor. On the other hand, the counts of the intratumor CD4+ T cells and NK cells were not related to the tumor growth.

It was observed that CD8+ T cells were induced in the tumor in the mice to which the spirulina extract was administered and in which an antitumor effect was observed.

### Example 6

In an experimental system similar to that of Example 1 in which MO5 tumor cells (1×10⁶ cells/200 µL D-PBS per animal) were subcutaneously injected to the shaved backs of C57BL/6 mice, oral administration of H₂O or the spirulina extract was conducted on day 6 and day 8 after the tumor transplantation.

On day 6, day 8 and day 10 after the tumor transplantation, 200 µg of a rat IgG2b isotype control antibody (Ab) (LTF-2) or an anti-PD-L1 antibody was intraperitoneally injected to the mice.

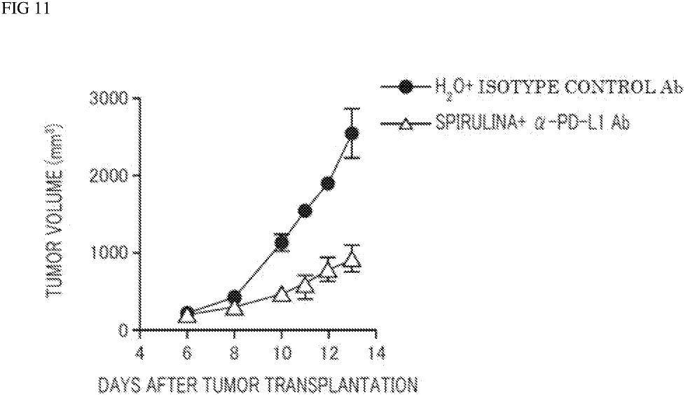

The results are shown in FIG. 11.

The results of the tumor growth in the mice to which H₂O + the isotope control Ab or the spirulina extract + the anti-PD-L1 Ab were orally administered are shown.

As shown in FIG. 11, when the anti-PD-L1 antibody and the spirulina extract were administered in combination, results of suppressing the swelling growth which were significantly superior to the results of the administration of H₂O and the isotype control Ab (LTF-2) without the anti-PD-L1 antibody were shown.

Accordingly, it was found that an immune checkpoint inhibitor (especially, a PD-1 pathway inhibitor such as an anti-PD-L1 antibody) can be used in combination with the spirulina extract for the purpose of suppressing the growth of a CTL-sensitive tumor.

As shown in the Examples above, it was found that a spirulina extract causes spontaneous activation of cytotoxic T lymphocytes (CTLs) and CTL-dependent regression of a tumor.

This means that the composition for CTL activation containing a spirulina extract of the invention can proliferate CTLs and can also suppress tumor growth.

Accordingly, a pharmaceutical composition and a food or a drink containing the composition for CTL activation of the invention can be effectively used for a pharmaceutical composition for CTL activation or a food or a drink for CTL activation for the purpose of CTL activation.

1. A composition for cytotoxic T lymphocyte (CTL) activation for proliferation of CTLs containing a spirulina extract.

2. The composition for CTL activation according to claim 1 which is for oral use.

3. A pharmaceutical composition for CTL activation containing the composition for CTL activation according to claim 1.

4. The pharmaceutical composition for CTL activation according to claim 3 which is used for treating any cancer of malignant melanoma (melanoma), non-small cell lung cancer, Hodgkin lymphoma, head and neck cancer, stomach cancer, kidney cancer and bladder cancer.

5. A combination of the pharmaceutical composition for CTL activation according to claim 3 and an immune checkpoint inhibitor.

6. A method for treating any cancer of malignant melanoma (melanoma), non-small cell lung cancer, Hodgkin lymphoma, head and neck cancer, stomach cancer, kidney cancer and bladder cancer in which the pharmaceutical composition for CTL activation according to claim 3 and an immune checkpoint inhibitor are used in combination.

7. A food or a drink for CTL activation containing the composition for CTL activation according to claim 2.
